(19) United States
(12) Patent Application Publication
(10) Pub. No.: US 2005/0181075 A1
(43) Pub. Date: Aug. 18, 2005

(54) Pharmacological enhancement and manufacturing method of antiviral compound

(76) Inventors: Jiao Gong, Arcadia, CA (US); Wei Jiang, Arcadia, CA (US)

Correspondence Address: JIAO GONG, 411 S. 3RD AVE #B, ARCADIA, CA, 91006, US

(21) Appl. No.: 10/718,350

(22) Filed: Nov. 21, 2003

(51) Int. Cl. A61K 35/78

(52) U.S. Cl. 424/725

(57) ABSTRACT

“Pharmacological enhancement and manufacturing method of the antiviral compound” can be categorized into the combined patent of the pharmacological activity as well as method of isolating and purifying the naturing material. Our product adopts 9 medicinal material which are processed strictly. The stable and high quality is ensured by the WLD resin adsorption and gas chromatography. The antiviral prevention and treatment is the most urgent but unsolved problem before the American doctors. The antiviral compound has an unexpected effect on a broad spectrum of viruses including RSV, Adenovirus type 3, Influenza A1 and A3. The antiviral effect on mouse Influenza A1 is very obvious. The definite efficacy in the treatment of acute pharyngitis and tonsillitis have been proved by the clinical trial which showed the total effective rate in the above two disease were 92.3% and 87.5% respectively.

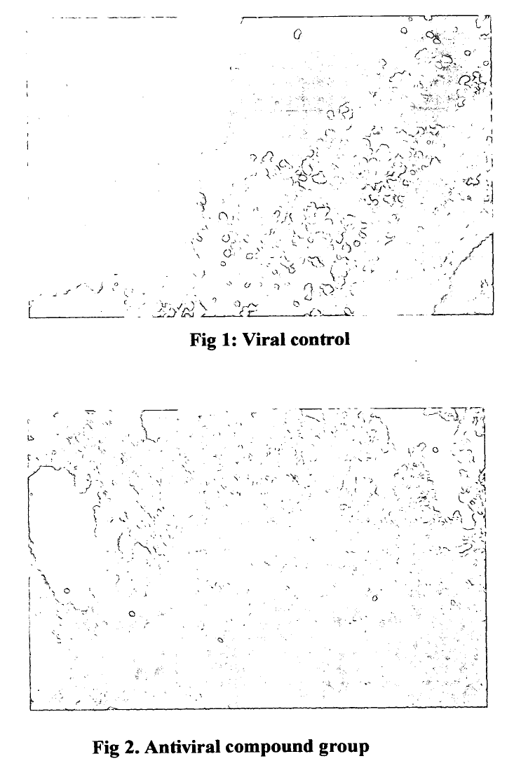

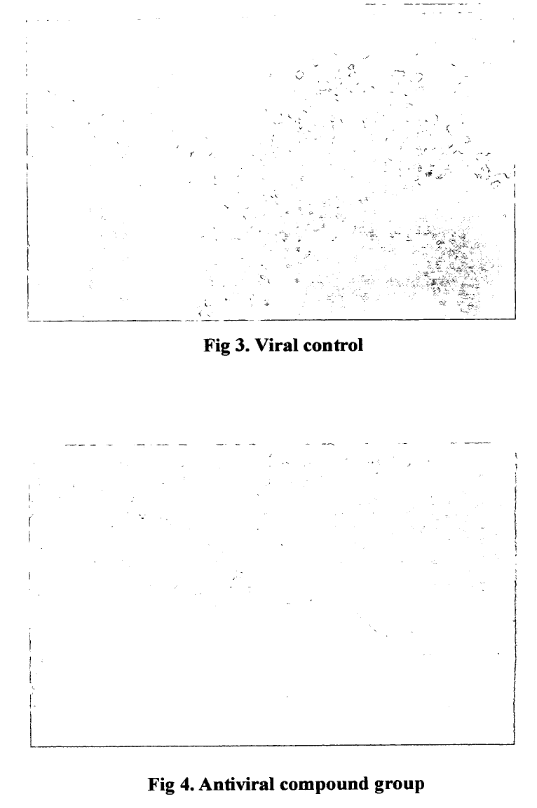

### BACKGROUND OF THE INVENTION

Viral infection and viral disease are spreading all over the world, posing threat to human's health. Governments have cooperated to wage along war against viral infection and viral disease. People are still deadly frightened on hearing the names of viral diseases such as AIDS, Dengue fever, rabies, polio. The SARS, a lethal pneumonia of mutant coronavirus, attacking China and other Asian countries, has caused a great panic worldwide. Warnings from WHO fuel the nervousness and panic. Looking back to the history of fighting against diseases, we are proud of our landslide victory over bacterial infection. However, the result of the fight against viral disease is still uncertain Scientists believe that the “game” is in a draw—neither human nor virus wins. Various retroviruses with small RNA or DNA molecule inside invade human body usually during the process of mutation. Unfortunately, it makes the doctors and patients fail in defending themselves.

As an antiviral compound, the invention adopts 9 natural medicinal materials which are processed strictly. Pharmacological experiments proved that our antiviral compound has the enhanced pharmacological activity, inhibiting and killing the Respiratory Syncytial Virus (RSV), Adenovirus type 3, Influenza Virus A₃, A₁. Our product has an unexpected effect on viral inhibition, and it is an invention of ours. Our invention is a combined patent of pharmacology as well as method of isolating and extracting natural material.

The case of Nelson V, Bowler, 626F 2d 853, 856, 206 USPQ 881, 883 (CCPA 1980) American court of Appeals for the Tariff and Patent claimed that it is good to publicize as much information about the pharmacological activity of known chemical, as possible. Once the chemical is known to the doctors, it is easier and quicker to Control the diseases or alleviate the symptoms. To make more information known to the public, it is necessary to encourage the researchers. To evaluate the claimed practicality, we should use the same de jure standard in the other areas. It should be noted that the practicality in the Patent Act should not be confused with the safety and efficacy requirements of FDA. (Food and Drug Administration). Evaluation of practicality is based on the pharmacological activity which is important for the treatment and prevention The antiviral compound meets all the practicality requirements in terms of treatment, prevention and pharmacology. In addition, it has creativity compound, judged from the definition given by American court of Appeals for the Tariff and Patent, can be categorized into the definition of “Chemicals”. Thus, pharmacological activity and manufacturing method, as a whole, is consistent with American Patent Act.

### BRIEF SUMMARY OF THE INVENTION

Andrew Chevallier, the author of Encyclopedia of Medicinal Plants, is of the opinion that the first choice for the disease treatment is language, then medicinal plant, and scalpel the last. Since ancient times, it has been believed that, medicinal plant can alleviate pain and cure disease. Even nowadays, 75 percent of the total drugs is from medicinal plant. The two major components of “Coca Cola” are extraction of plant. For centuries, scientists all over the world have set up their own system to develop medicinal plant and their application. Some methods seem reasonable and practical. However, all methods are designed to cure disease, reduce pain, and improve cure disease, reduce pain, and improve living standard.

Among all the diseases, viral infection is the most terrible. Every ten years sees a massive mutant Influenza virus epidemic and every five years a minor epidemic. Worse still, people can neither predict the Influenza virus mutation trend nor estimate the detailed mutation information but they have to confront with the Influenza outbreak. The virus can invade the human body, damage the immune system, or even cause more massive injuries. Killing virus is the most urgent but unsolved problem for the American doctors. Our antiviral compound adopts the following medicinal materials. The manufacturing method of antiviral compound with enhanced pharmacological activity can be described as “extracting by distillation and decoction; absorbing the working components with WLD resin column; eluting by 65% ethanol at the room temperature; mixing the filtrate and the oil-water mixer; spray-drying at 85° C.; gas chromatography to ensure the quality.

Pharmacological experiments of our antiviral compound indicated that the maximum dilution ratio and inhibition index of different virus are as follows: RSV, 1:256, 4, respectively; Adenovirus type 3, 1:512, 8, respectively; Influenza virus A1, 1:16, 8 respectively. The result of the chicken embryo experiments are as follows: for the influenza virus A1, the maximum dilution ratio of the viral inhibition 1:2, inhibition index: 2; for the influenza virus A3, 1:4, 2 respectively. The results of pharmacological experiments indicated that for the group of 12.5 g/kg/d, LW:BW (lung weight:body weight):8.09±0.17, inhibition rate:22.87%; for the group of 25.0/kg/d, LW:BW 7.98±0.23, inhibition rate: 23.92%. The inhibition rate is directly proportional to the concentration. All the experiments indicate that the antiviral compound has obvious effect on the viral inhibition. According to the result of viral granule specific fluorescence assay, the normal control is negative, but viral control positive. As compared with 64.35% of the viral group, the antiviral compound group is 20.12%. Thus, there is significant difference. The microscopic examination report that for the viral group, the pathological lesion is serious, presenting the neutrocyte infiltration and necrotizing tissue in the bronchial cavity as well as pink staining proteinous fluid in the alveolar space; for the antiviral group, the lesion is mild, presenting little fluid and few white cells in few alveoli and bronchioles. It indicates that the antiviral compound can inhibit the proliferation of Influenza virus A1 (see Table 4 and 6).

To determine the pharmacological activity of the antiviral compound, the clinical observation has been conducted among the patient groups of acute phayyngitis as well as acute tonnitis. For the acute pharyngitis group the total apparently effective rate is 78.0%, total effective rate 92.3%; for the acute tonnitis group, 74.2% and 87.5% respectively (see Table 5). Clinical trial has proved that the pharmacological activity of the antiviral compound is reliable.

### BRIEF DESCRIPTION OF THE SEVERAL VIEWS OF THE DRAWING CONTENT

Part 1 Components of the antiviral compound

Part 2 Raw material sources of the antiviral compound

Part 3 Manufacturing method and process flow of the antiviral compound.

Part 4 Pharmacological enhancement of the antiviral compound

Part 5 Clinical Observation of the antiviral compound efficacy among the patients with acute pharyngitis as well as acute tonnitis

Part 6 Microscopic observation of the mouse lung specimen: the serious lesion in the viral group but mild in the antiviral group.

Part 1 Components of the antiviral compound (see attached table 1)

Part 2 Raw material sources of the antiviral compound (see attached table 2)

Part 3 Manufacturing method and process flow of the antiviral compound.

(see attached table 3)

Part 4 Pharmacololgical enhancement of the antiviral compound
1. Subject: (batch No.20302)
2. Virus: Influenza virus A1, A3, RSV, and Adenovirus type 3.
3. Cell: Hela cell, Vero cell.
4. Chicken embryo.
5. Experimental (animal: inbred mice, 14-16 g)
   Method
   I. Experiment In Vitro

1. Cytopathic Effect of Virus

Drug Cytotoxicity Determination (Toxic-free Load Determination): Micro Cell Culture Antiviral compound containing solutions at different concentration (test drug and positive control) as well as the same volume of maintaining solution was added into the Hela (or Vero) cell culture wells (0.2 ml/well, 4 wells/concentration level). The cell was cultured in the 5% CO₂ incubator at 37° C. for 72 hours. The toxic-free viral load was defined as the minimum dilution rate for cytopathic change.

Effect of the Drug on the Cytopathic Change:
(1) 100 TCID 50 virus containing solution (RSV or Adenovirus type 3) was added in the Hela cell culture (0.1 ml/well). Incubation for 1 hour. After rinsing the cell culture well, 0.2 ml antiviral compound containing solution (diluted by maintaining solution) was added, 4 well per concentration level. Viral control and cell control were set. The cells were cultured in 5% CO₂ incubator at 37° C. The cell culture was observed under the inverted microscope. Once the cytopathic effect appeared, the maximum dilution ratio was recorded and the inhibition index was calculated.(Inhibition index=Maximum dilution ratio/toxic-free viral load)

(2) Influenza A1 Virus Infection Test: Except for the Vero cell, all the procedures were the same as above. Because the cytopathic change was mild, Red Cell Adsorption Test was use instead. 72 hours after culture with the antiviral compound, 0.1 ml 0.08% guinea rat red cells was added in the wells. After 30 minutes, the red blood adsorption was observed under the microscope to determine the maximum dilution rate and calculate the inhibition index.

2. Effect of the drug on the Influenza Virus A1 and A3 proliferation in the Chicken Embryo.

Toxicity Determination of the drug to the Chicken Embryo: 0.2 ml antiviral compound at different concentration was injected into the allantoic cavity of chicken embryo (10 days). 4 chicken embryos per concentration level, the control (normal saline) was set. Incubation at 35° C. for 72 hours. The living embryo number was counted. The minimum dilution rate which did not cause the death of chicken embryo was defined as the toxic-free viral load.

Effect of the drug on the Influenza Virus in the Chicken embryo: The 15LD50 Influenza virus containing solution mixed with the same volume of antiviral compound containing solution. 0.2 ml mixed solution (at different concentration) was added in four chicken embryos. Viral control was set. Incubation at 35° C. for 72 hours, 4° C. overnight. The allantoic fluid was collected for the blood clotting test. The maximum dilution rate at which the blood clotting was not observed was recorded to calculate the inhibition rate.

II Experiment in Vivo.

1. Effect of the drug on the mouse viral pneumonia caused by Influenza Virus.

The mice were divided randomly into antiviral, viral and control group, 10 mice per group, male and female equal in number. 0.4 ml antiviral solution (at different dilution concentration) was given through the gastric intubation 1 day before the viral infection and lasting for 5 days. For the control, the same volume of normal saline was given instead. 15LD50, the Influenza virus A1 strain (lung adaption type) was inoculated via nasal drop. The mice were sacrificed 96 hours after infection The lungs of the mice were weighed and the LW:BW was calculated (LW:BW=lung weight/body weight). The date was analysed by T-test.

2. The Effect of the Drug on the viral granule proliferation Viral Granule Specific Fluorescence Percentage:

Mouse anti-Influenza virus antibody was added to the cryosection of the mouse lung specimen. After 40 minutes for reaction, the anti-mouse IgG fluorescence antibody was add. After another 40 minutes for reaction, the slide was rinsed and dried in the air. The viral granule fluorescence percentage was calculated after observing under the fluorescence microscope.

3. Histopathology: As the routine H.E. staining and microscopic examination.

### Result

1. The results of antiviral experiments with RSV, Adenovirus type 3 and Influenza Virus A1. With the infection of 100 TCID 50 virus, the maximum dilution ratio and the inhibition index were as follows: the RSV 1:256 (antiviral dosage 7.81 mg/mil), 4 respectively; Adenovirus 3, 1:512(3.90 mg/ml), 8 respectively; Influenza virus A1, 1:16 (125 mg/ml), 8 respectively.

The maximum dilution ratio of the antiviral compound in case of Influenza Virus A1 was 1:16, and the inhibition index was 8(see Table 1)

Table 1: Antiviral of the drug on RSV, Adenovirus type 3 and Influenza Virus A1 (see attached table 4)

2. Antiviral Effect of the drug on Influenza virus in the chicken embryo.

The results indicated that the maximum ratio was 1:2 (for Influenza Virus A1) and 1:4 (for influenza virus A3) respectively (table 2).

Table 2. Antiviral effect of the drug on Influenza virus in the chicken embryo. (see attached table 5)

3. Antiviral effect of the drug on the Influenza Virus in the mice.

(1) 96 hours after infection, the mice were sacrificed and the lung was weigned to calculated the LW:BW (See Table 3).

Table 3. Antiviral effect of the drug on the Influenza virus A1 in mice. (see attached table 6)

The results showed that for the group with the dosage of 12.5 g/kg/d, LW:BW 8.09±0.17 and inhibition rate 22.87%; for the group with the dosage of 25.0 g/kg/d, LW:BW 7.98±0.23 and inhibition rate 23.92%. The antiviral effect is directly proportional to the dosage. In contrast to viral control, there was significant different (p<0.001). It indicated that the antiviral drug has obvious effect on the inhibition of Influenza Virus A1 proliferation.

(2) Viral granule specific fluorescence percentage (in the bronchioles): The normal control was negative, the viral control was positive with the fluorescence percentage 64.35%; and the antiviral group was 20.12% with significant difference in contrast to the viral control (p<0.01).

(3) Histopathology: For the viral control, the lesion was serious, presenting the neutrophiles and necrotizing cells in the branchioles as well as the pink-staining proteinous fluid in the alveolar space. However, the antiviral group showed mild changes with only few white cells and little fluid. It indicates that the antiviral compound is effect on the inhibition of Influenza A1 proliferation in mice.

### Conclusion

1. The results of the cell culture in vitro proved that the antiviral compound has the inhibitive effect on RSV, Adenovirus type 3, Influenza virus A1 with the inhibition index of 4, 8, 8 respectively.

2. The results of the chicken embryo experiment proved that the antiviral compound has the inhibitive effect on Influenza virus A1 and A3 with the inhibition index of 2 and 4 respectively.

3. The mouse experiment in vivo includes the following parameters: LW:BW, viral inhibition ratio, specific fluorescence percentage and histopathological examination. There is significant difference between the antiviral compound group and the control. The antiviral compound has the inhibitive effect on Influenza Virus A1.

Part 5 Clinical trial of the antiviral compound in the treatment of acute pharyngitis and acute tonsillitis

Table 1 Comprehensive efficacy comparison (see attached table 7)

Treatment group Total apparent effect rate 78.0%, Total effect rate 92.3%.

Table 2 Comprehensive efficacy comparison (see attached table 8)

Total apparent effect rate of the treatment group 74.2%, total effective rate 87.5%

Table 3 Main Symptoms and signs comparison before and after treatment (see attached table 9)

For the treatment group, the main symptoms and signs were improved and the effective rate was obvious.

Table 4 Main Symptoms and signs comparison before and after treatment (see attached table 10)

For the treatment group, the main symptoms and signs were improved and the effective rate was obvious.

Table 5. The relationship between acute pharyngitis and treatment (see attached table 11)

Table 6. The relationship between acute tonsillitis and treatment (see attached table 12)

Table 7. Effect start time comparison (day) (see attached table 13)

Table 8. Effect start time comparison. (see attached table 14)

Table 9. The relationship between acute pharyngitis course and effect (see attached table 15)

There is significant different between 1-day-course group and 2-day-course group (Rank test, P<0.01): The efficacy of the antiviral drug in the 1-day-course group is greater than that in the 2-day-course group.

Table 10. The relationship between acute tonsillitis course and effect (see attached table 16)

There is no significant difference between 1, 2, 3 day course group. (Rank test, P<0.05). The efficacy of different treatment group is quite similar.

### Conclusion

The results of clinical trial indicated that the antiviral compound has definite efficacy in the treatment of acute pharyngitis and tonsillitis. The total effective rate and total apparent effect rate for the acute pharyngitis are 92.3% and 78.0% respectively, and for the acute tonsillitis, 87.5% and 74.2% respectively.

Part 6 Histopathological Examination

The lesion is severe for the viral control but mild for the antiviral compound group. (see attached table 17)

### DETAILED DESCRIPTION OF THE INVENTION

1. “Pharmacological enhancement and manufacturing method of the antiviral compound” can be categorized into the combined patent of pharmacology as well as method of isolating and purifying natural material. Characterized with the unexpected efficacy and talent inspiration, it has met the standard of patent. Our product adopts the following: The manufacturing method can be described ad follows: mixing every 100 kg Comu Bubaci and Widus Vespae with 1000 kg water; decocting for 4 hours; filtrating, collecting dregs and repeating; collecting the two filtrates; distilling other raw material at 100° C. for 4 hours; collecting the volatile oil-water mixer; decocting the dregs of 100 kg with 800 kg water for 3 hours and repeating; collecting the filtrates; absorbing via WLD resin column; eluting by 65% ethanol; mixing all the filtrates and oil-water mixer; spray-drying at 85° C.; component analysis by the gas chromatography to ensure the quality (see Part 3).

2. The results of the cell culture in vitro indicate that the antiviral compound has inhibitive effect on RSV Adenovirus type 3, and Influenza virus A1. The inhibition index of RSV and Adenovirus type 3 are 4 and 8 respectively.

3. The results of the chicken embryo experiment indicate that the antiviral compound has inhibitive effect on Influenza A1 and A3. The inhibition index are 2 and 4 respectively.

4. The mouse experiment in vivo includes the following parameters: LW:BW, inhibition rate, specific fluorescence rate, and pathological changes. There is significant difference between the antiviral compound group and the control. Thus, the antiviral compound is effective on the inhibition of mouse Influenza virus A1 (see Table 4 and 6).

5. Clinical trial showed the efficacy of the antiviral compoud in the treatment of acute pharyngitis and tonsillitis. The total effective rate and total apparent effect rate for the acute pharyngitis group are 92.3% and 78.0% respectively, and for the acute tonsillitis group, 87.5% and 74.2% respectively. Thus, the pharmacological activity of the antiviral compound is reliable (see Table 5).

TABLE 1

| | |
|---|---|
| Radix Scutellariae | 12% |
| Fructus Forsythiae | 20% |
| Herbs Schizone Petae | 15% |
| Flos Chrysanthemi | 15% |
| Radix Scrophulariae | 15% |
| Cornu Bubaci | 10% |
| Radixet Rhizoma Rhei | 4.5% |
| Spina Gleditsiae | 4.5% |
| Widus Vespae | 4% |
| Total | 100% |

TABLE 2

| | | | | | |
|---|---|---|---|---|---|
| 1 | Radix Scutellariae | 2 | Fructus Forsythiae | 3 | Herbs Schizone Petae |
| | Labiatae Scutellaria | | Oleaceae Forsythia | | Labiatae Schizonepeta |
| | baicalensis Georgi(root) | | Suspensa(Thumb) VahL. [symbol] | | tenuifolia Brig (falling |
| | | | (fruit) | | branches and leaves) |
| 4 | Flos Chrysanthemi | 5 | Radix Scrophulariae | 6 | Cornu Bubaci |
| | Composilae Chrysanthemum | | Scrophulariaceae | | Bovidae Bubalus bubalis |
| | morifolium | | Scrophularia ningpoensis | | Linnaeus(horn) |
| | Ramat(capitulum) | | HemsL.(root) | | |
| 7 | Radixet Rhizoma | 8 | Spina Gleditsiae | 9 | Widus Vespae |
| | Rhei | | Leguminosae Gleditisa | | Vespidae Polistes |
| | Polygonaceae Rheum | | Sineusis Lam(thorns) | | Olivaceous(DeGeer) (adult) |
| | PalmatumL.(root and stem) | | | | |

(attached table 3)

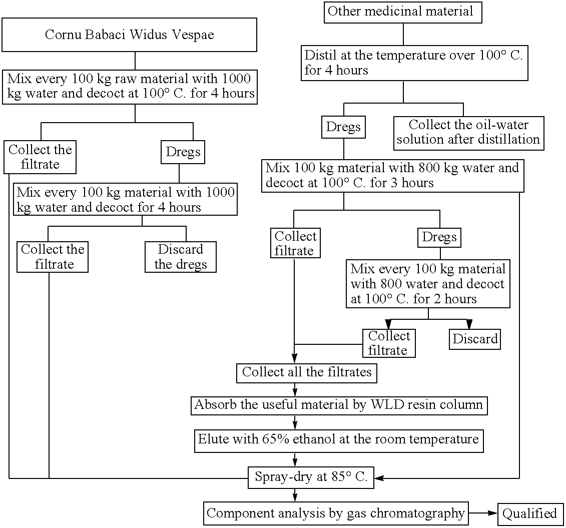

TABLE 4

| | | | Adenovirus | A1 (Influenza |
|---|---|---|---|---|
| Name | | RSV | type 3 | Virus A1) |
| Antiviral | Toxic-free Viral Load | 1:64 | 1:64 | 2 |
| compound | (including the | (31.25 mg/ml) | (31.25 mg/ml) | (4000 mg/ml) |
| | drug dosage mg/ml) | | | |
| | Maximum Dilution Ratio | 1:256 | 1:512 | 1:16 |
| | (including the drug | (7.81 mg/ml) | (3.90 mg/ml) | (125.00 mg/ml) |
| | dosage mg/ml) | | | |
| | Inhibition Index | | | |

TABLE 5

| | | Influenza | Influenza |
|---|---|---|---|
| Name | RSV | Virus A1 | Virus A3 |
| Antiviral | Toxic-free Viral Load | 1 | 1 |
| compound | (including the drug | (2000 mg/ml) | (2000 mg/ml) |
| | dosage mg/ml) | | |
| | Maximum Dilution Ratio | 1:2 | 1:4 |
| | (including the | (1000 mg/ml) | (500 mg/ml) |
| | drug dosage mg/ml) | | |
| | Inhibition Index | | |

TABLE 6

| | Dosage | LW:BW | Inhibition | |
|---|---|---|---|---|
| Group | (g/kg/d) | (X) | rate (%) | P value |
| Viral control | — | 10.49 ± 0.45 | | |
| Normal control | — | 7.86 ± 0.32 | | |
| Antiviral | | | 12.01 | <0.05 |
| Compound | | | | |
| Antiviral | 6.875(12.5) | 8.09 ± 0.17 | 22.87 | <0.001 |
| Compound | | | | |
| Drug | 13.75(25.0) | 7.98(0.23) | 23.92 | <0.001 |
| dosage(g/kg/d) | | | | |

TABLE 7

| | | | | | | | | | | | Total | | | |
|---|---|---|---|---|---|---|---|---|---|---|---|---|---|---|
| | | | | | Apparent | | | | | | apparent | | Total effect | |
| | | | Cured | | effect | | Effect | | Invalid | | effect rate | | rate | |
| Disease | Group | n | Cases | % | Cases | % | Cases | % | Cases | % | Cases | % | Cases | % |
| Acute | Treatment | 182 | 79 | (43.4) | 63 | (34.6) | 26 | (14.3) | 14 | (7.7) | 142 | (78.0) | 168 | (92.3) |
| pharyngitis | group | | | | | | | | | | | | | |

Rank test:
U = 3.24
P < 0.01

TABLE 8

| | | | | | | | | | | | Total | | | |
|---|---|---|---|---|---|---|---|---|---|---|---|---|---|---|
| | | | | | Apparent | | | | | | apparent | | Total effect | |
| | | | Cured | | effect | | Effect | | Invalid | | effect rate | | rate | |
| Disease | Group | n | Cases | % | Cases | % | Cases | % | Cases | % | Cases | % | Cases | % |
| Acute | Treatment | 120 | 56 | (46.7) | 33 | (27.5) | 16 | (13.3) | 15 | (12.5) | 89 | (74.2) | 105 | (87.5) |
| tonsillitis | group | | | | | | | | | | | | | |

Rank test:
U = 3.65
P < 0.001

TABLE 9

| | | | Pharyngalgia | | | Retro- | | Para- |
|---|---|---|---|---|---|---|---|---|
| | | | with | pharyngalgia | | pharyngeal | | pharyngeal |
| | | | swallowing | involved | Pharyngeal | lympho- | uvular | swellingness |
| Group | | Pharyngalgia | pain | with ear | congestion | proliferation | congestion | and redness |
| Treatment | Before | 53 | 106 | 23 | 130 | 129 | 32 | 92 |
| group | treatment | | | | | | | |
| | After | 5 | 3 | 5 | 29 | 27 | 5 | 8 |
| | treatment | | | | | | | |
| | Effective | 90.6% | 97.2% | 78.3% | 77.7% | 79.1% | 84.4% | 91.3% |
| | rate | | | | | | | |

TABLE 10

| | | | Pharyngalgia | | | | |
|---|---|---|---|---|---|---|---|
| | | | with | pharyngalgia | | | Purulent |
| | | | swallowing | involved | Tonsil | Swollen | secretion |
| Group | | Pharyngalgia | pain | with ear | congestion | tonsil | on the tonsil |
| Treatment | Before | 32 | 67 | 21 | 84 | 77 | 31 |
| group | treatment | | | | | | |
| | After | 3 | 9 | 6 | 25 | 14 | 5 |
| | treatment | | | | | | |
| | Effective | 90.6% | 86.6% | 71.4% | 70.2% | 81.8% | 83.9% |
| | rate | | | | | | |

TABLE 11

| | | | | | | | | | | | | Total |
|---|---|---|---|---|---|---|---|---|---|---|---|---|
| | | | | | | | | | | | Total | apparent |
| | | | | | Apparent | | | | | | effective | effect |
| | | | Cured | | effect | | Effect | | Invalid | | rate | rate |
| Group | Severity | n | Cases | % | Cases | % | Cases | % | Cases | % | % | % |
| Treatment | Mild | 47 | 32 | (68.1) | 10 | (21.3) | 5 | (10.6) | 0 | (0) | 100 | 89.4 |
| group | Medium | 110 | 45 | (40.9) | 43 | (39.1) | 15 | (13.6) | 7 | (6.4) | 93.6 | 80.0 |
| | Severe | 25 | 2 | (8.0) | 10 | (40.0) | 6 | (24.0) | 7 | (28.0) | 72.0 | 48.0 |

TABLE 12

| | | | | | | | | | | | | Total |
|---|---|---|---|---|---|---|---|---|---|---|---|---|
| | | | | | | | | | | | Total | apparent |
| | | | | | Apparent | | | | | | effective | effect |
| | | | Cured | | effect | | Effect | | Invalid | | rate | rate |
| Group | Severity | n | Cases | % | Cases | % | Cases | % | Cases | % | % | % |
| Treatment | Mild | 28 | 22 | (78.6) | 6 | (21.4) | 0 | (0) | 0 | (0) | 100 | 100 |
| group | Medium | 67 | 25 | (37.4) | 22 | (32.8) | 10 | (14.9) | 10 | (14.9) | 85.1 | 70.2 |
| | Severe | 25 | 9 | (36.0) | 5 | (20.0) | 6 | (24.0) | 5 | (20.0) | 80.0 | 56.0 |

TABLE 13

| Disease | Group | Cases (effect) | X ± SD |
|---|---|---|---|
| Acute pharyngitis | Treatment group | 168 | 1.39 ± 0.66 |

TABLE 14

| Disease | Group | Cases (effect) | X ± SD |
|---|---|---|---|
| Acute tonsillitis | Treatment group | 105 | 1.78 ± 0.90 |

TABLE 15

| Group | n | Efficacy | One day | Two days |
|---|---|---|---|---|
| Treatment group | 79 | Cured | 48 | 31 |
| | 63 | Apparent effect | 23 | 40 |
| | 26 | Effect | 10 | 16 |
| | 14 | Invalid | 6 | 8 |

TABLE 16

| Group | n | Efficacy | One day | Two days | Three days |
|---|---|---|---|---|---|
| Treatment | 56 | Cured | 17 | 20 | 19 |
| group | 33 | Apparent effect | 7 | 18 | 8 |
| | 16 | Effect | 4 | 7 | 5 |
| | 15 | Invalid | 4 | 4 | 7 |

1. I claim that “the Pharmacological Enhancement and Manufacturing Method of the Antiviral Compound” is my invention. My antiviral compound adopts the following as raw material:
The process flow of manufacturing can be described as “mixing every 100 kg coma babaci and widus vespae with 1000 kg water; decocting for 4 hours; filtrating and repeating it (dregs); collecting the two filtrates; distilling other raw material for 100 kg dregs with 800 kg water; decocting for 3 hours; filtrating and repeating it for the dregs (but decocting time was 2 hours); adsorbing with wld resin; eluting with 65% ethanol; mixing all the filtrates; spray-drying at 85 c; gas chromatography for component analysis to ensure its quality:
I believe that my inventions (including Antiviral compound) are effective in the inhibition of RSV, Adenovirus type 3, Influenza Virus A1 and A3 especially for the mouse Influenza Virus A1. Clinical trial proves its efficacy in the treatment of acute pharyngitis and acute tonsillitis.
